(19) United States
(12) Patent Application Publication
(10) Pub. No.: US 2015/0142141 A1
(43) Pub. Date: May 21, 2015

(54) ELECTRONIC DEVICE AND REMOTE CONTROL METHOD

(71) Applicant: Kabushiki Kaisha Toshiba, Tokyo (JP)

(72) Inventors: Nobuhide Okabayashi, Tachikawa (JP); Takuya Ootani, Ome (JP)

(21) Appl. No.: 14/607,787

(22) Filed: Jan. 28, 2015

Related U.S. Application Data
(63) Continuation of application No. PCT/JP2013/058616, filed on Mar. 25, 2013.

(51) Int. Cl. G05B 15/02; A61B 5/00; A61B 5/024; A61B 5/01

(52) U.S. Cl. CPC G05B 15/02; A61B 5/01; A61B 5/4809; A61B 5/024; A61B 5/681; A61B 5/6843
USPC 700/83

(57) ABSTRACT

According to one embodiment, an electronic device includes a first detector, a memory and circuitry. The first detector detects either attachment of the electronic device to a human body or removal of the electronic device from a human body. The memory is configured to store control data. The circuitry executes a process foe transmitting a command for performing a process specified by the control data to at least one external device specified by the control data either when the attachment of the electronic device is detected or when the removal of the electronic device is detected.

### CROSS REFERENCE TO RELATED APPLICATIONS

This application is a Continuation Application of PCT Application No. PCT/JP2013/058616, filed Mar. 25, 2013, the entire contents of which are incorporated herein by reference.

### FIELD

Embodiments described herein relate generally to an electronic device and a remote control method.

### BACKGROUND

Recent years have seen the development of electronic devices called wearable devices attachable to the users. Many wearable devices can provide the users wearing the devices with various types of information.

In addition, a start has been made on developing techniques for controlling external devices by using a wearable device in response to the operation of the wearable device.

However, to control an external device, the user must operate the wearable device; and to control a plurality of external devices, the user must perform on the wearable device individual operations for each of the external devices.

### BRIEF DESCRIPTION OF THE DRAWINGS

A general architecture that implements the various features of the embodiments will now be described with reference to the drawings. The drawings and the associated descriptions are provided to illustrate the embodiments and not to limit the scope of the invention.

FIG. 1 is an exemplary view showing the outline of a remote control process which is executed by an electronic device according to an embodiment.

FIG. 2 is an exemplary view for explaining the outline of the electronic device according to the embodiment.

FIG. 3 is an exemplary view showing an example of the system configuration of the electronic device according to the embodiment.

FIG. 4 is an exemplary block diagram showing the relationship of a plurality of components provided in the electronic device according to the embodiment.

FIG. 5 is an exemplary flowchart showing a procedure of a remote control process which is executed when the electronic device of the embodiment is attached to the user.

FIG. 6 is an exemplary flowchart showing a procedure of a remote control process which is executed when the electronic device of the embodiment is removed from the user.

FIG. 7 is an exemplary flowchart showing a procedure of a remote control process which is executed while the electronic device of the embodiment is attached to the user.

FIG. 8 is an exemplary view for explaining control data indicating the content of a process which should be executed when the electronic device of the embodiment is attached or removed.

FIG. 9 is an exemplary view for explaining another type of control data indicating the content of a process which should be executed while the electronic device of the embodiment is attached to the user.

FIG. 10 is an exemplary view for explaining the outline of a remote control process which is executed by the electronic device of the embodiment when the user goes out/comes back home.

FIG. 11 is an exemplary block diagram showing the relationship of components related to the remote control process which is executed when the user goes out/comes back home.

FIG. 12 is an exemplary flowchart showing a procedure of a remote control process which is executed when the electronic device of the embodiment goes out of a predetermined area.

FIG. 13 is an exemplary flowchart showing a procedure of a remote control process which is executed when the electronic device of the embodiment enters a predetermined area.

FIG. 14 is an exemplary flowchart showing a procedure of a user authentication process which is executed by the electronic device according to the embodiment.

### DETAILED DESCRIPTION

Various embodiments will be described hereinafter with reference to the accompanying drawings.

In general, according to one embodiment, an electronic device includes a first detector, a memory and circuitry. The first detector detects either attachment of the electronic device to a human body or removal of the electronic device from a human body. The memory is configured to store control data. The circuitry executes a process foe transmitting a command for performing a process specified by the control data to at least one external device specified by the control data either when the attachment of the electronic device is detected or when the removal of the electronic device is detected.

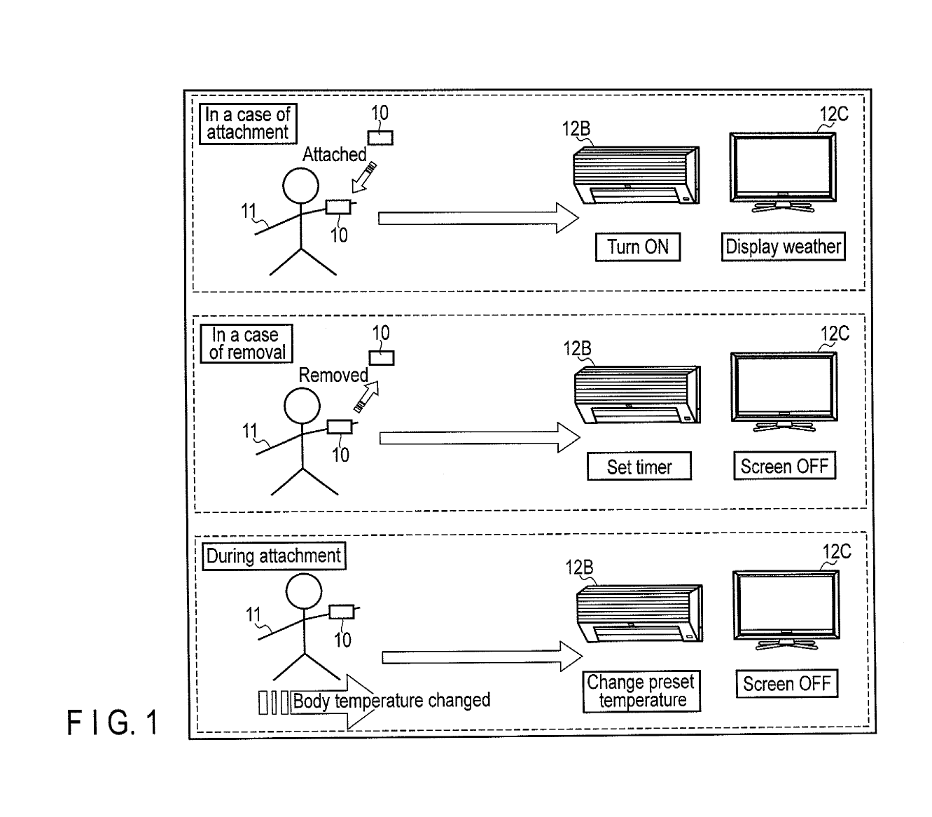

With reference to FIG. 1, the outline of an electronic device according to an embodiment will be described. The electronic device is realized as a wearable device 10 attachable to the human body. The wearable device 10 has a function for providing a user 11 wearing the wearable device 10 with various types of information, a device cooperation function for operating in cooperation with various external devices such as electronic devices at home, etc. The device cooperation function includes a function for automatically remote-controlling various external devices.

The wearable device 10 comprises various built-in sensors and is configured to detect biological data of the human body (user 11). The wearable device 10 is also configured to detect attachment of the wearable device 10 to the human body (user 11) and removal of the wearable device 10 from the human body (user 11).

In the present embodiment, the wearable device 10 is configured to remotely control a plurality of in-home information devices (external devices 12B and 12C) as a whole upon attachment of the wearable device 10 to the user 11 or removal of the wearable device 10 from the user 11. The wearable device 10 is also configured to remotely control external device 12B and external device 12C as a whole upon change of biological data of the user 11 (as a trigger).

FIG. 1 shows the outline of a remote control process which is executed by the wearable device 10. The remote control process is executed when the user 11 wears the wearable device 10 and removes the wearable device 10. FIG. 1 also shows the outline of the present embodiment during the attachment of the wearable device 10. In the present embodiment, the period “during the attachment of the wearable device 10” does not refer to the period from start to end of the action of the user 11 for wearing the wearable device 10 and refers to the state in which the wearable device 10 has been attached to the user 11.

When the wearable device 10 is attached to the user 11, the wearable device 10 performs remote control for turning external device 12B (air conditioner) on (in other words, for changing the state of external device 12B to an on-state). The wearable device 10 also performs remote control for displaying weather information in external device 12C (TV). The weather information is, for example, information of a program which broadcasts weather information on the day. Thus, the attachment of the wearable device 10 to the user 11 enables the plurality of external devices 12B and 12C to be controlled (operated) as a whole. Specifically, the attachment of the wearable device 10 to the user 11 enables the states of the plurality of external devices 12B and 12C to be changed. For example, the wearable device 10 is attached to the user 11 when the user 11 wakes up.

When the wearable device 10 is removed from the user 11, the wearable device 10 performs remote control for setting the timer of external device 12B. For example, when the user 11 sets the timer of external device 12B at bedtime, the user 11 only needs to remove the wearable device 10 to set the timer of external device 12B. When the wearable device 10 is removed from the user 11, the wearable device 10 performs remote control for turning the screen of external device 12C off (in other words, for changing the state of the screen of external device 12C to an off-state). Thus, the user 11 can turn the screen of external device 12C off by merely removing the wearable device 10. The user 11 does not need to conduct an operation for turning the screen of external device 12C off. In this manner, the external devices can be operated as a whole when the wearable device 10 is removed from the user 11.

While the wearable device 10 is attached to the user 11 (in other words, during the attachment), the wearable device 10 performs remote control for changing the preset temperature of external device 12B in accordance with the change of biological data of the user 11. The wearable device 10 also performs remote control for turning the screen of external device 12C off. For example, when the user 11 feels hot, the preset temperature of external device 12B decreases. When the user 11 falls asleep, the body temperature of the user 11 decreases. In response to the decrease in the body temperature of the user 11, the screen of external device 12C is turned off. Thus, even when the user 11 falls asleep with the wearable device 10 attached, the screen of external device 12C can be turned off. In this manner, the external devices can be controlled as a whole while the wearable device 10 is attached to the user 11 (in other words, during the attachment).

Thus, the wearable device 10 is configured to automatically change the operating environment of an external device around the user 11. For example, the wearable device 10 is configured to change the brightness of lighting by adjusting the illuminance of lighting (an external device) at home (not shown in FIG. 1).

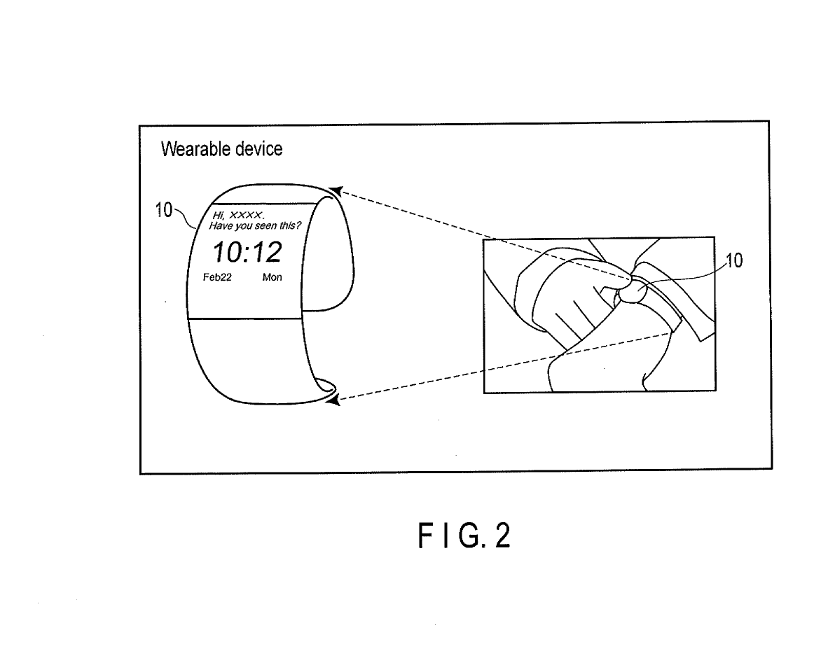

Now, the outline of the wearable device 10 of the present embodiment will be described with reference to FIG. 2.

Hereinafter, this specification assumes that the wearable device 10 is realized as a wristwatch-like device attachable to the arm of the human body. The wearable device 10 of the present embodiment is not limited to a wristwatch-like device and may be, for example, a glasses-like device or an earphone-like device wearable on the ears of the user. In sum, the wearable device 10 of the present embodiment is a device which the user is not conscious of wearing.

The wearable device 10 comprises sensors configured to measure various types of biological data of the person wearing the wearable device 10. For example, the sensors are configured to measure vein pattern, body temperature, bioelectrical impedance, heart rate and blood pressure. The wearable device 10 authenticates the user based on biological data. The user is authenticated based on, for example, whether or not the biological data of the user 11 registered in the wearable device 10 agrees with or corresponds to the biological data measured by the above sensors. When the biological data agree with or correspond to each other, the authentication of the user is determined as successful. When the authentication of the user is successful, the wearable device 10 is configured to (remotely) control an external device. Moreover, the wearable device 10 has a wireless communication function. The wearable device 10 is configured to control an external device by wirelessly communicating with the external device. The external device is, for example, an in-home information device such as the air conditioner 12B, the television (TV) 12C or a refrigerator.

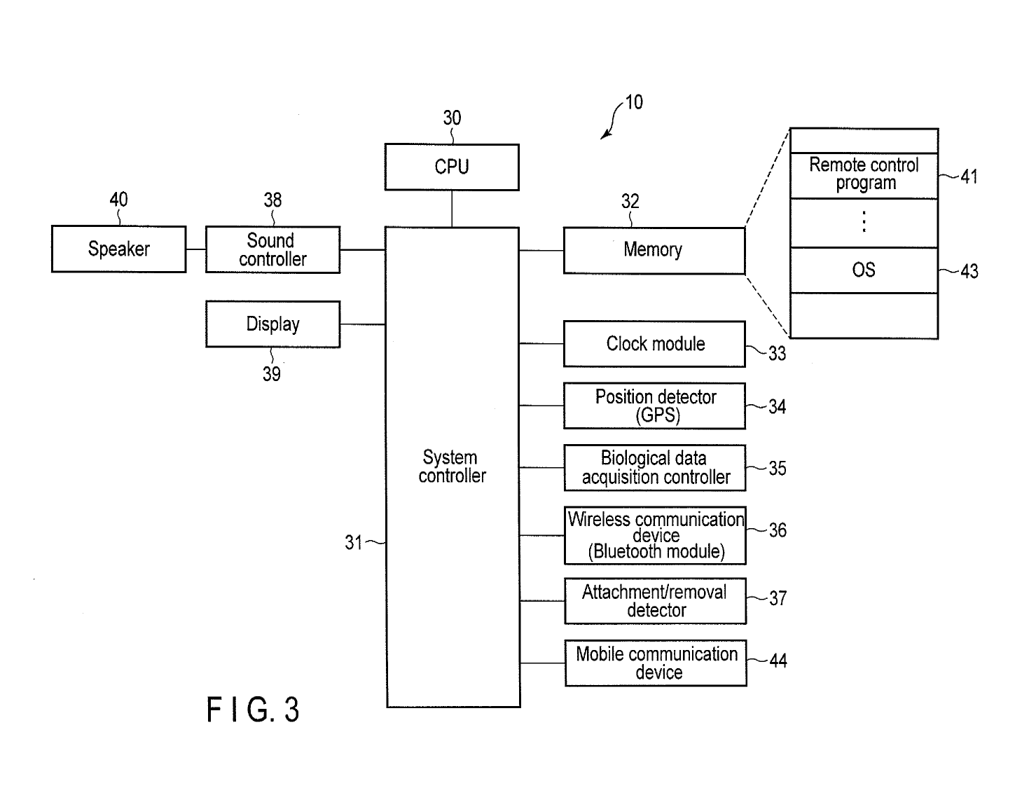

Now, the system configuration of the wearable device 10 will be described with reference to FIG. 3.

The wearable device 10 comprises a CPU 30, a system controller 31, a memory 32, a clock module 33, a position detector (GPS) 34, a biological data acquisition controller 35, a wireless communication device (Bluetooth (registered trademark) module) 36, an attachment/removal detector 37, a sound controller 38, a display 39, a speaker 40 and the like.

The CPU 30 is a processor configured to control the operation of each component of the wearable device 10. The CPU 30 includes circuitry (processing circuitry). The CPU 30 executes an operating system (OS) 43 and various application programs loaded into the memory 32. The application programs include a remote control program 41.

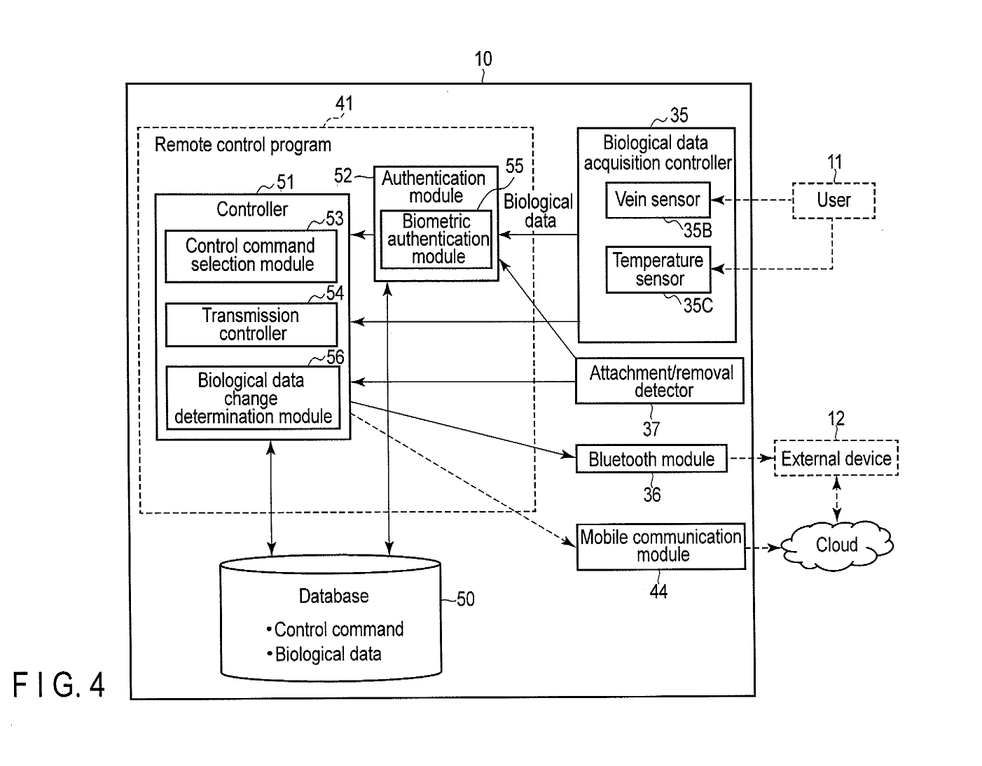

The remote control program 41 is a program for remotely operating the above external devices. The detail of the program is explained later with reference to FIG. 4. Briefly speaking, the remote control program 41 is configured to operate an external device by transmitting a control command (remote control command) for controlling the external device to the external device upon attachment of the wearable device 10 to the user or removal of the wearable device 10 from the user. The control command is transmitted to the external device as a command for performing a certain process.

The system controller 31 is a bridging device configured to connect the CPU 30 and each component. The system controller 31 comprises a built-in memory controller configured to control the access to the memory 32. The system controller 31 may comprise a built-in display controller configured to control the display 39 of the wearable device 10. Further, the system controller 31 has a function for performing communication with the sound controller 38.

The sound controller 38 is a sound source device and outputs the audio data to be reproduced to the speaker 40. The wireless communication device (Bluetooth module) 36 is a wireless communication device configured to perform wireless communication by using, for example, Bluetooth (registered trademark). The wireless communication device (Bluetooth module) 36 may use wireless communication which is different from Bluetooth (registered trademark) and enables short-distance communication with an external device.

The clock module 33 is a module configured to measure the current time. The position detector 34 obtains the current position of the wearable device 10. For example, the position detector 34 obtains the longitude and latitude indicating the current position of the wearable device 10. For example, the position detector 34 conforms to the global positioning system (GPS).

The biological data acquisition controller 35 includes one or more sensors configured to measure (obtain) biological data of the user 11. The attachment/removal detector 37 detects attachment of the wearable device 10 to the user (human body) 11 or removal of the wearable device 10 from the user (human body) 11. Specifically, the attachment/removal detector 37 detects closing of the buckle of the wearable device 10 as attachment of the wearable device 10 to the user (human body) 11 and detects opening of the buckle as removal of the wearable device 10 from the user (human body) 11.

A mobile communication device 44 performs wireless communication by using a mobile telephone communication network. For example, the mobile communication device 44 performs wireless communication conforming to the 3G communication standard. The mobile communication device 44 only has to perform wireless communication conforming to a standard which is different from the Bluetooth module 36. The mobile communication device 44 may perform wireless communication using a communication network which is different from a mobile telephone communication network.

Now, the configuration of software executed by the wearable device 10 will be described, referring to FIG. 4.

The wearable device 10 comprises the biological data acquisition controller 35, the Bluetooth module 36, the attachment/removal detector 37, the remote control program 41, a storage 50 and the like.

The attachment/removal detector 37 detects attachment or removal of the wearable device 10. The attachment/removal detector 37 notifies a controller 51 and an authentication module 52 that the wearable device 10 is attached or removed.

The biological data acquisition controller 35 comprises a vein sensor 35B and a temperature sensor 35C. The biological data acquisition controller 35 obtains biological data of the user 11. The vein sensor is a sensor configured to read the shape of blood vessels of the user. The temperature sensor 35C is a sensor configured to measure the body temperature of the user.

The biological data acquisition controller 35 sends the biological data sensed by, for example, the vein sensor 35B or the temperature sensor 35C to the controller 51 and the authentication module 52.

The biological data acquisition controller 35 is configured to sense the body temperature of the user by using the temperature sensor 35C, etc., during the attachment of the wearable device 10.

The authentication module 52 comprises a biometric authentication module 55. The biometric authentication module 55 starts biometric authentication upon detection of attachment of the wearable device 10 by the attachment/removal detector 37. The biometric authentication module 55 compares the user's biological data measured by the biological data acquisition controller 35 (sensor) to the biological data (registered biological data) stored in the storage 50. The biometric authentication module 55 determines whether or not the measured biological data corresponds to the registered biological data, or in other words, whether or not the feature of the measured biological data matches (corresponds to/agrees with) that of the registered biological data. When the biometric authentication module 55 has succeeded in the biometric authentication, that is, when the measured biological data matches the registered biological data, the authentication module 52 notifies the controller 51 that the user has been authenticated.

The controller 51 comprises a control command selection module 53, a transmission controller 54 and a biological data change determination module 56. The controller 51 transmits a control command to external device 12 in response to the notification of detection of attachment or removal of the wearable device 10 from the attachment/removal detector 37. That is, the controller 51 transmits a control command (command) for performing a certain process to the external device 12 either when the attachment of the wearable device 10 is detected or when the removal of wearable device 10 is detected.

The control command is, for example, a command for changing the state of an external device configured to operate in cooperation with the wearable device 10 by remote control.

In the storage (memory) 50, control data indicating the content of a process which should be executed when attachment or removal of the wearable device 10 is detected is stored. Specifically, in the storage 50, control data including control commands is stored as a database. The control data includes first control data indicating the content of the process which should be executed when attachment of the wearable device 10 is detected, second control data indicating the content of the process which should be executed when removal of the wearable device 10 is detected, and control data indicating the content of the process which should be executed when the user's biological data is changed.

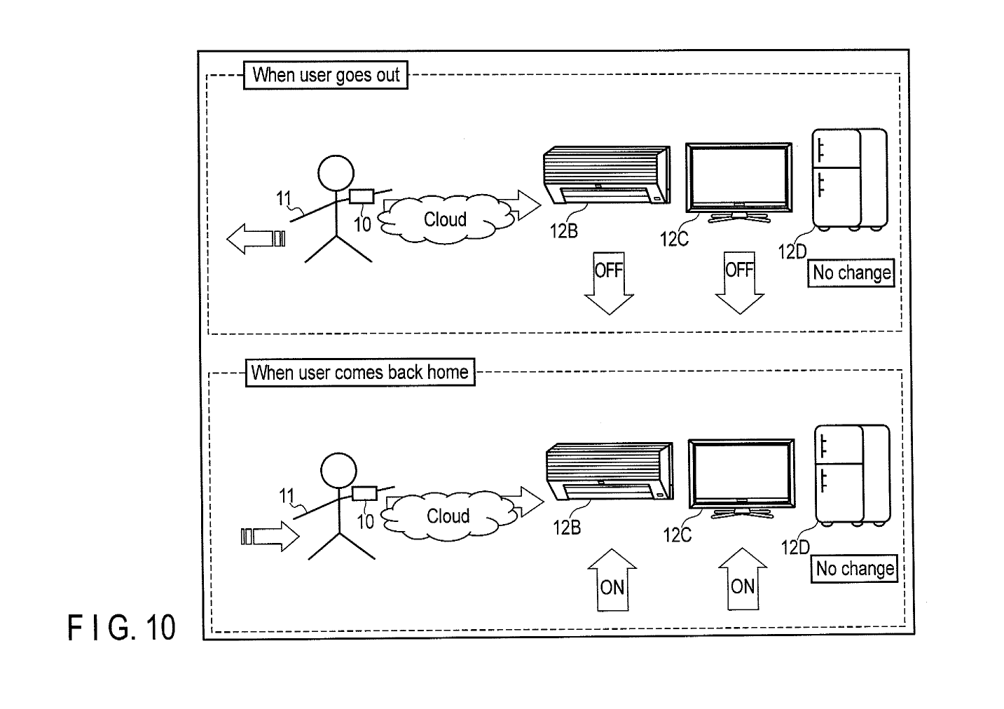

The controller 51 determines whether or not the wearable device 10 is present in a predetermined area (the user's house) in which at least one external device is present based on the position data detected by the position detector 34 of FIG. 3. When the wearable device 10 is present in the predetermined area, the controller 51 enables the function for transmitting a control command. When the wearable device 10 is not present in the predetermined area, the controller 51 disables the function for transmitting a control command. The detail of this process is explained later with reference to FIG. 10.

The control command selection module 53 refers to the database stored in the storage 50 and selects (determines) a control command to be transmitted to external device 12 based on the data from the attachment/removal detector 37 or the biological data obtained by the biological data acquisition controller 35. The transmission controller 54 transmits the control command selected by the control command selection module 53 to external device 12 via the Bluetooth module 36. The transmission module 54 may transmit the control command to external device 12 via a cloud system connected by the mobile communication device 44. The cloud system is, for example, a system including a server which manages a plurality of external devices.

The biological data change determination module 56 determines whether or not the biological data is changed based on the biological data obtained by the biological data acquisition controller 35. For example, the biological data change determination module 56 determines whether or not the user's body temperature sensed by the temperature sensor 35C exceeds a predetermined threshold which is set in advance. When the biological data change determination module 56 determines that the biological data is changed, the control command selection module 53 selects a control command corresponding to the change in the biological data based on the database stored in the storage 50.

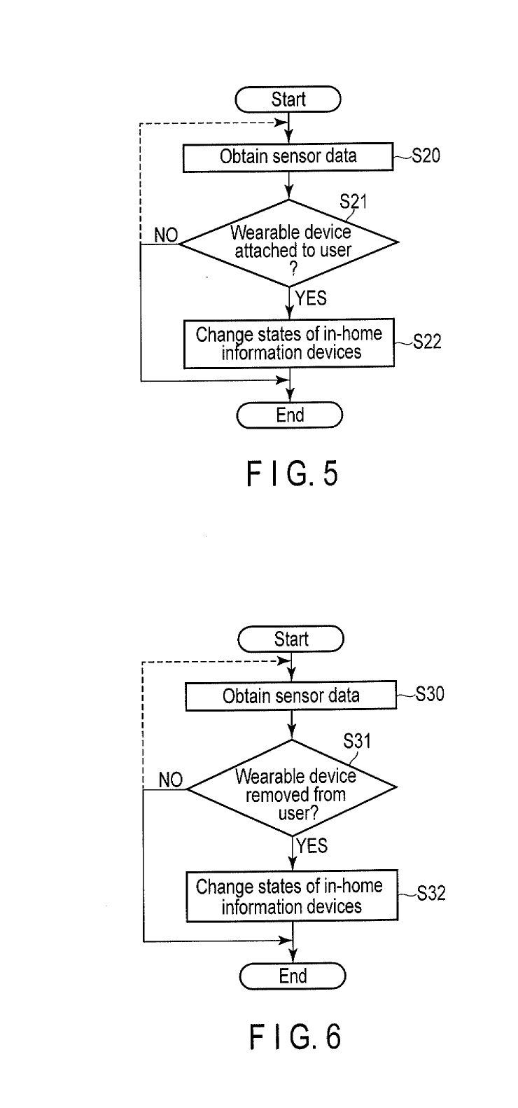

This specification turns to the explanation of a procedure of a remote control process which is executed when the wearable device 10 is attached to the user with reference to FIG. 5.

The remote control program 41 starts a remote control process upon detection of attachment or removal of the wearable device 10 by the attachment/removal detector 37. Subsequently, the remote control program 41 obtains biological data which is sensor data of the vein sensor 35B, etc., from the biological data acquisition controller 35 (step S20). The remote control program 41 starts user authentication based on the obtained biological data. When the user is successfully authenticated, the remote control program 41 determines whether or not attachment of the wearable device 10 is detected by the attachment/removal detector 37 (step S21). The user authentication in step S20 may not be conducted. Specifically, after the remote program 41 obtains the biological data from the biological data acquisition controller 35, step S21 may be started without conducting the user authentication. In this manner, the remote control program 41 may recognize the user by obtaining the biological data from the biological data acquisition controller 35 without conducting the user authentication. When the remote control program 41 determines that attachment of the wearable device 10 is detected by the attachment/removal detector 37 (YES in step S21), the remote control program 41 transmits control commands to external devices 12 through communication between the wearable device 10 and external devices 12. The control commands to be transmitted are, for example, commands for changing the state of each of the external devices 12 to operating state (on-state, etc.). For example, one of the control commands to be transmitted is a control command for changing the TV channel relative to external device 12C (TV). In this manner, the states of external devices 12 are changed.

When the remote control program 41 determines that attachment of the wearable device 10 is not detected by the attachment/removal detector 37 in step S21 (NO in step S21), the remote control process which is executed when the wearable device 10 is attached to the user is terminated as shown in FIG. 5. However, for example, when attachment or removal of the wearable device 10 is detected by the attachment/removal detector 37 and the biological data of the user is obtained by the biological data acquisition controller 35, the controller 51 may detect attachment or removal of the wearable device 10. In this case, the remote control program 41 may wait for the biological data acquisition controller 35 to obtain the user's biological data again when the remote control program 41 determines that attachment of the wearable device 10 is not detected by the attachment/removal detector 37 in step S21 of FIG. 5 (NO in step S21).

Now, this specification explains a procedure of a remote control process which is executed when the user removes the wearable device 10 with reference to FIG. 6.

The remote control program 41 starts a remote control process upon detection of attachment or removal of the wearable device 10 by the attachment/removal detector 37. Subsequently, the remote control program 41 obtains biological data which is sensor data of the vein sensor 35B, etc., from the biological data acquisition controller 35 (step S30). The remote control program 41 starts user authentication based on the obtained biological data. When the user is successfully authenticated, the remote control program 41 determines whether or not removal of the wearable device 10 is detected by the attachment/removal detector 37 (step S31). The user authentication in step S30 may not be conducted. Specifically, after the remote control program 41 obtains the biological data from the biological data acquisition controller 35, step S31 may be started without conducting the user authentication. In this manner, the remote control program 41 may recognize the user by acquiring the biological data from the biological data acquisition controller 35 without conducting the user authentication. When the remote control program 41 determines that removal of the wearable device 10 is detected by the attachment/removal detector 37 (YES in step S31), the remote control program 41 transmits control commands to external devices 12 through communication between the wearable device 10 and external devices 12. The control commands to be transmitted are, for example, commands for changing the state of each of the external devices 12 to a stopped state (off-state, etc.). In this manner, the state of each external device 12 is changed.

When the remote control program 41 determines that removal of the wearable device 10 is not detected by the attachment/removal detector 37 in step S31 (NO in step S31), the remote control process which is executed when the user removes the wearable device 10 is terminated as shown in FIG. 6. However, for example, when attachment or removal of the wearable device 10 is detected by the attachment/removal detector 37 and the biological data of the user is obtained by the biological data acquisition controller 35, the controller 51 may detect attachment or removal of the wearable device 10. In this case, the remote control program 41 may wait for the biological data acquisition controller 35 to obtain the user's biological data again when the remote control program 41 determines that attachment of the wearable device 10 is not detected by the attachment/removal detector 37 in step S31 of FIG. 6 (NO in step S31).

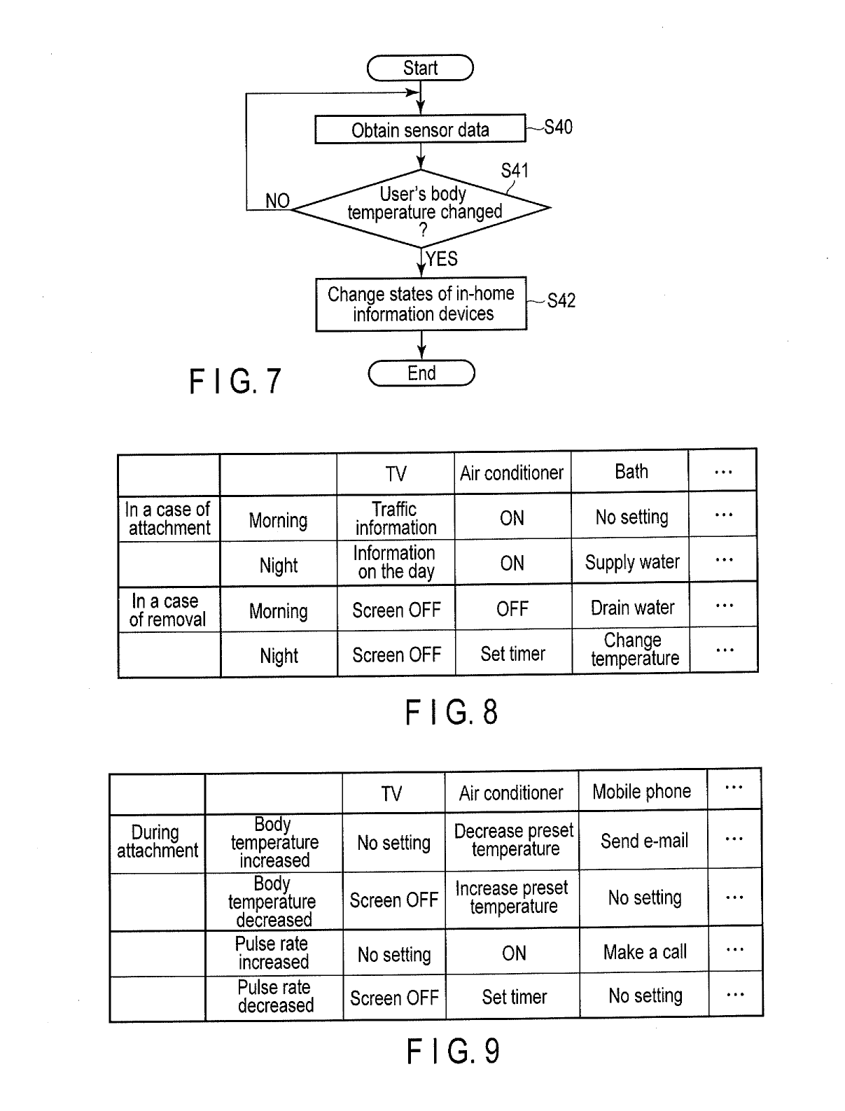

Now, this specification explains a procedure of a remote control process which is executed when the biological data of the user is changed during the attachment of the wearable device 10, with reference to FIG. 7.

While the wearable device 10 is attached to the user, the remote control program 41 performs the process shown in FIG. 7. The remote control program 41 obtains the data of the body temperature of the user as biological data (sensor data) sensed by the biological data acquisition controller 35 (step S40). Subsequently, the biological data change determination module 56 determines whether or not the obtained data related to the body temperature of the user is changed (step S41). When the biological data change determination module 56 determines that the obtained data related to the body temperature of the user is changed (YES in step S41), the remote control program 41 transmits control commands corresponding to the change in the body temperature data to external devices 12 through communication between the wearable device 10 and external devices 12. The control commands to be transmitted are, for example, commands which are associated with the change in the biological data obtained by the biological data acquisition controller 35. In this manner, the state of each of external devices 12 is changed. When the biological data change determination module 56 does not determine that the obtained data related to the body temperature of the user is changed (NO in step S41), the remote control program 41 obtains biological data of the user again from the biological data acquisition controller 35.

This specification explains an example of the database stored in the wearable device 10 with reference to FIG. 8.

The detail of the example is explained with reference to FIG. 8. The database shown in FIG. 8 shows the content of a control command corresponding to each of external devices when the wearable device 10 is attached (hereinafter, referred to as in a case of attachment) and when the wearable device 10 is removed (hereinafter, referred to as in a case of removal).

The external devices are a TV, an air conditioner, a device provided in a bath (hereinafter, referred to as a bath setting device), etc. The wearable device 10 comprises the clock module 33 as explained above. Therefore, the wearable device 10 is configured to transmit different control commands to each of the external devices in accordance with the time.

In a case of attachment in the morning, for example, the user may wear the wearable device 10 after the user woke up. In this case, a control command for displaying traffic information on the TV screen is transmitted to the TV and further, a control command for turning the air conditioner on (changing the state of the air conditioner to an on-state) is transmitted to the air conditioner. In this case, with regard to the bath setting device, a control command for changing the state of the device is not set.

In a case of attachment at night, for example, the user may wear the wearable device 10 after the user came back home. In this case, a control command for displaying the information on the day on the TV screen is transmitted to the TV, and further, a control command for turning the air conditioner on (changing the state of the air conditioner to an on-state) is transmitted to the air conditioner. Moreover, a control command for storing hot water in the bath (changing the state of the bath to a water-supplying state) is transmitted to the bath setting device.

In a case of removal in the morning, for example, the user may remove the wearable device 10 when the user goes out. In this case, a control command for turning the screen off (changing the state of the screen to a screen-off state) is transmitted to the TV so that no image is displayed on the TV screen, and further, a control command for turning the air conditioner off (changing the state of the air conditioner to an off-state) is transmitted to the air conditioner. Moreover, a control command for draining the hot water of the bath (changing the state of the bath to a water-draining state) is transmitted to the bath setting device.

In a case of removal at night, for example, the user may remove the wearable device 10 when the user goes to bed or takes a bath. In this case, a control command for turning the TV screen off (changing the state of the screen to a screen-off state) is transmitted to the TV so that no image is displayed on the screen, and further, a control command for setting the duration of time before turning the air conditioner off (changing the state of the air conditioner to a timer-set state) is transmitted to the air conditioner. Moreover, a control command for changing the temperature of the hot water of the bath (changing the state of the bath to a temperature-change state) is transmitted to the bath setting device.

In a case of attachment or removal, a control command may be transmitted to each of external devices as explained with reference to FIG. 8. However, a control command may be transmitted to a single external device.

Now, this specification explains another example of the database stored in the wearable device 10, referring to FIG. 9.

The database of FIG. 9 shows the content of a control command corresponding to each of external devices while the wearable device 10 is attached (during the attachment).

When the user's body temperature sensed by the temperature sensor 35C increases during the attachment, a control command for turning down the preset temperature of the air conditioner is transmitted to the air conditioner. Further, a control command for sending e-mail is transmitted to a mobile phone which is an external device. The increase in the body temperature of the user could indicate abnormal change in the physical condition of the user. The mobile phone sends e-mail to the family, etc., in response to the increase in the body temperature of the user wearing the wearable device 10. In this manner, the family can know abnormal change in the physical condition of the user wearing the wearable device 10. (This function is called a watch-over function).

During the attachment, the body temperature of the user may decrease. For example, the body temperature of the user may decrease after the user falls asleep went to bed. In this case, a control command for turning the TV screen off (changing the state of the screen to a screen-off state) is transmitted to the TV, and further, a control command for turning up the preset temperature of the air conditioner is transmitted to the air conditioner.

The biological data acquisition controller 35 may comprise a sensor configured to measure the pulse rate (heart rate). As shown in FIG. 9, the pulse rate may increase during the attachment. For example, the increase in the pulse rate may cause the body temperature to go up and make the user feel hot, or the increase in the pulse rate may indicate abnormal change in the physical condition of the user. In this case, a control command for turning the air conditioner on (changing the state of the air conditioner to an on-state) is transmitted to the air conditioner, and further, a control command for calling the family, etc., is transmitted to the mobile phone.

When the pulse rate decreases, for example, when the user falls asleep during the attachment, a control command for changing the TV screen to an off-state is transmitted to the TV, and further, a control command for changing the air conditioner to a timer-set state is transmitted to the air conditioner.

During the attachment, a control command may be transmitted to each of external devices as explained with reference to FIG. 9. However, a control command may be transmitted to a single external device.

The databases shown in FIG. 8 and FIG. 9 may be databases generated based on the user's personal data stored in a cloud system. The personal data includes, for example, data of a control command relative to external device 12 set by the user 11 in advance. The wearable device 10 may obtain the personal data from the cloud system and generate the databases shown in FIG. 8 and FIG. 9 based on the obtained personal data.

The databases shown in FIG. 8 and FIG. 9 may be registered in the storage 50, and the user 11 may select a database from the registered databases.

The remote control program 41 may have a leaning function for changing the content of the database stored in the storage 50 in accordance with the use state of the user 11. For example, the user 11 may frequently change the TV to an on-state in the morning. In such a case, the remote control program 41 may set the information related to the control command to be transmitted to the TV in the morning during the attachment as shown in FIG. 8 such that the information indicates an on-state.

Now, this specification explains the outline of other functions provided in the wearable device 10 of the present embodiment, referring to FIG. 10.

As other functions, the following three functions are provided.

First function: This function is configured to switch the mode of the wearable device 10 between a first mode and a second mode upon detection of exit of the wearable device 10 from a predetermined area or entry of the wearable device 10 into the predetermined area. In the first mode, attachment or removal of the wearable device 10 is detectable. In the second mode, attachment or removal of the wearable device 10 is not detectable. In the first mode, the function which causes the wearable device 10 to transmit a control command to external device 12 is enabled. In the second mode, the function which causes the wearable device 10 to transmit a control command to external device 12 is disabled.

Second function: This function is configured to change the state of an external device present in a predetermined area to a predetermined state upon detection of exit of the wearable device 10 from the predetermined area or entry of the wearable device 10 into the predetermined area.

Third function: This function is configured to save the state of an external device present in a predetermined area upon detection of exit of the wearable device 10 from the predetermined area, and restore the state of the external device present in the predetermined area based on the saved state of the external device upon detection of entry of the wearable device 10 into the predetermined area.

This specification explains the detection of exit of the wearable device 10 from the predetermined area or entry into the predetermined area in the first to third functions in more detail. The wearable device 10 obtains the position data of the wearable device 10 by using the GPS, etc., when the user 11 wearing the wearable device 10 goes out. The wearable device 10 is configured to detect exit of the user 11 from the predetermined area or entry into the predetermined area based on the obtained position data. A polling process may be applied from an external device at regular intervals in order to determine whether or not the wearable device 10 is present in the predetermined area. For example, when a sensor configured to detect the wearable device 10 is provided in the entrance of the user's house, the user 11 can switch the mode of the wearable device 10 between the first mode and the second mode by merely going through the entrance.

The first function is explained first.

The first function is configured to perform the above-described detection of attachment or removal of the wearable device 10 only in a predetermined area. When the first function is used, for example, it is possible to prevent external device 12 present in a predetermined area such as the user's house from being operated outside the house. The mode of the wearable device 10 is switched to the first mode upon detection of entry of the user 11 into the predetermined area. Therefore, outside the predetermined area, the user 11 can use the wearable device 10 as a normal wristwatch which does not have the function for operating an external device. In the predetermined area, the user 11 can use the wearable device 10 as a wristwatch which has the function for operating an external deice.

Now, this specification explains the second and third functions with reference to FIG. 10.

When exit from or entry into the predetermined area is detected (sensed), the wearable device 10 is configured to change the state of an information device (external device) present at home (in the predetermined area).

Specifically, when the user 11 goes out of the house, the wearable device 10 detects exit of the wearable device 10 from the house which is the predetermined area. The wearable device 10 changes the state of an external device which presumably does not need to operate (which is presumably unnecessary) in the house while the user 11 is away from the house. Specifically, as shown in FIG. 10, the wearable device 10 turns external device 12B (air conditioner) off (changes the state to a power-off state) and turns the screen of external device 12C (TV) off (changes the state to a screen-off state). Thus, the wearable device 10 is configured to turn an unnecessary external device off. Since external device 12D (refrigerator) does not seem to be unnecessary, the state of external device 12D is not changed. As shown in FIG. 10, the wearable device 10 may change the state of external device 12 via a cloud system.

The wearable device 10 is configured to save the state of an external device present in the house when the user 11 goes out. Specifically, when the user 11 goes out, the wearable device 10 saves, in the wearable device 10, the state of an external device which seems to be unnecessary before the state is changed. Specifically, when the user 11 goes out, the wearable device 10 saves, in the wearable device 10, information indicating that external device 12B is in an on-state, the screen of external device 12C is in an on-state and external device 12D is in an on-state. The on-state of external device 12D means, for example, a state in which the refrigerator is turned on. When the user 11 goes out, the wearable device 10 may save, in a server on a cloud system, the state of an external device which seems to be unnecessary before the state is changed.

When the user 11 comes back home, the wearable device 10 is configured to restore the state which was saved when the user 11 left home with respect to an external device present in the house. For example, as shown in FIG. 10, when the user 11 comes back home, the wearable device 10 changes the state of external device 12B to an on-state, changes the state of the screen of external device 12C to an on-state and changes the state of external device 12D to an on-state. In this manner, the states of external devices 12 before the user 11 goes out can be restored as a whole. Since the state of external device 12D was not changed from an on-state when the user left home, the wearable device 10 does not need to change the state of external device 12D.

For example, when the user 11 comes back home, reproduction of the recorded program which had been viewed by the user 11 by using a recording/reproduction device which is an external device before the user 11 left home may be restarted (not shown in FIG. 10). Specifically, the wearable device 10 transmits a control command for suspending the reproduction of the recorded program to the recording/reproduction device when the user 11 goes out. When the user 11 comes back home, the wearable device transmits a control command for reproducing the recorded program from the reproduction position in which the reproduction was suspended to the recording/reproduction device. In this manner, the reproduction of the recorded program may be restarted.

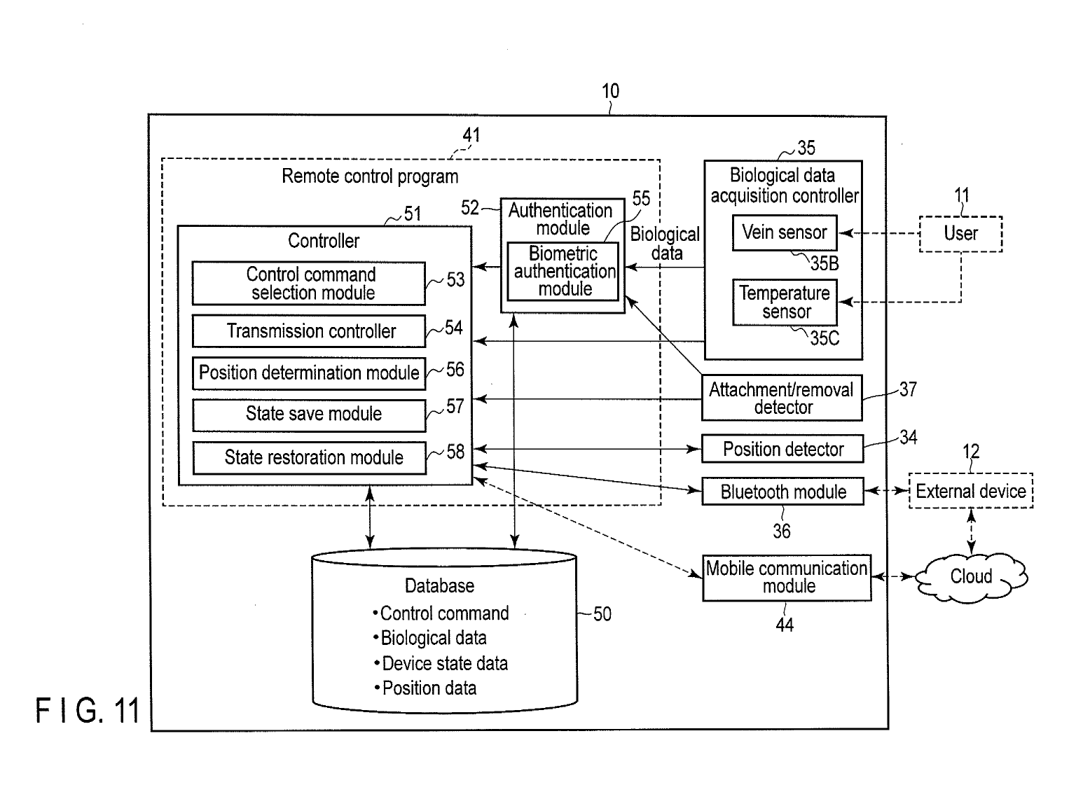

Referring to FIG. 11, this specification explains the system configuration of the wearable device 10 of the present embodiment having the three functions which are explained above with reference to FIG. 10. The explanation of the configurations or functions described with reference to FIG. 4 is omitted.

The wearable device 10 comprises the position detector 34, etc. The position data of the wearable device 10 detected by the position detector 34 is transmitted to the controller 51. The controller 51 comprises the position determination module 56, a state save module 57, a state restoration module 58 and the like. In the storage 50, for example, device state data and registered position data (position data) are stored. The device state data is data indicating the state of an external device. The registered position data is position data related to a predetermined area. Specifically, the registered position data is data which is registered in advance and which is related to, for example, a position from a predetermined external device by a predetermined distance.

The position determination module 56 determines the position of the wearable device 10 based on the position data obtained by the position detector 34 and the registered position data stored as a database in the storage 50. Specifically, the position determination module 56 reads the registered position data from the storage 50 and compares the registered position data which has been read to the position data obtained by the position detector 34. When the position determination module 56 determines that the position data obtained by the position detector 34 agrees with or corresponds to the registered position data, detection of exit of the wearable device 10 from the predetermined area or entry of the wearable device 10 into the predetermined area is determined. In other words, detection of presence of the wearable device 10 in the predetermined area or presence of the wearable device 10 outside the predetermined area is determined.

For example, when position data related to the longitude and latitude obtained by the GPS is used, the range of a predetermined area corresponds to an area surrounded by a plurality of longitudes and latitudes. When the longitudes and latitudes in the area surrounded by the plurality of longitudes and latitudes agree with the obtained longitude and latitude, it is possible to determine that the wearable device 10 is present in the predetermined area. When the longitudes and latitudes in the area surrounded by the plurality of longitudes and latitudes do not agree with the obtained longitude and latitude, it is possible to determine that the wearable device 10 is present outside the predetermined area. The GPS obtains position data at regular time intervals. Therefore, when the position determination module 56 determines that the wearable device 10 is present in the predetermined area and then determines that the wearable device 10 is present outside the predetermined area, the position determination module 56 is able to determine the detection of exit of the wearable device 10 from the predetermined area. On the other hand, when the position determination module 56 determines that the wearable device 10 is present outside the predetermined area and then determines that the wearable device 10 is present in the predetermined area, the position determination module 56 is able to determine the detection of entry of the wearable device 10 into the predetermined area.

When the position determination module 56 determines the detection of exit of the wearable device 10 from the predetermined area, the state save module 57 obtains external device state data indicating the state of external device 12 from external device 12 via the Bluetooth module 34 or the mobile communication device 44. The state save module 57 stores the obtained external device state data in the storage 50. The state save module 57 stores the obtained external device state data in the storage 50 and notifies the transmission controller 54 that a control command for changing the state of unnecessary external device 12 explained above should be transmitted to external device 12.

Specifically, in the storage 50, another type of control data (hereinafter, referred to as third control data) indicating the content of the process which should be executed when the wearable device 10 is detected as having exited from the predetermined area is stored. The state save module 57 saves data indicating the current state of at least one external device which is the control target specified by the third control data based on the third control data. In addition, the transmission controller 54 transmits a control command for setting at least one external device which is the control target specified by the third control data to the state specified by the third control data.

The transmission controller 54 determines the control command to be transmitted to external device 12 in response to the notification. The transmission controller 54 determines the control command for making the state of external device 12 appropriate based on the external device state data obtained by the state save module 57 from external device 12. The transmission module 54 transmits the determined control command to external device 12 via the Bluetooth module 34 or the mobile communication device 44. The external device state data is data indicating the state of external device 12 before the state of an unnecessary external device is changed. The external device state data is, for example, data indicating the on-state or off-state of external device 12.

When the position determination module 56 determines the detection of entry of the wearable device 10 into the predetermined area, the state restoration module 58 reads the external device state data stored in the storage 50 from the storage 50 and restores the state of external device 12 based on the read external device state data. The state restoration module 58 notifies the transmission controller 54 that a control command should be transmitted to the external device and transmits the read external device state data to the transmission controller 54 in order to restore the state of external device 12. The transmission controller 54 determines a control command for restoring the state of external device 12 based on the external device state data obtained from the state restoration module 58. For example, the transmission controller 54 transmits a control command corresponding to the state of the external device shown by the obtained external device state data.

Specifically, the transmission controller 54 transmits a control command for restoring at least one external device which is the control target specified by the third control data to the original state based on the data indicating the current state stored by the state storage module 57 regarding at least one external device.

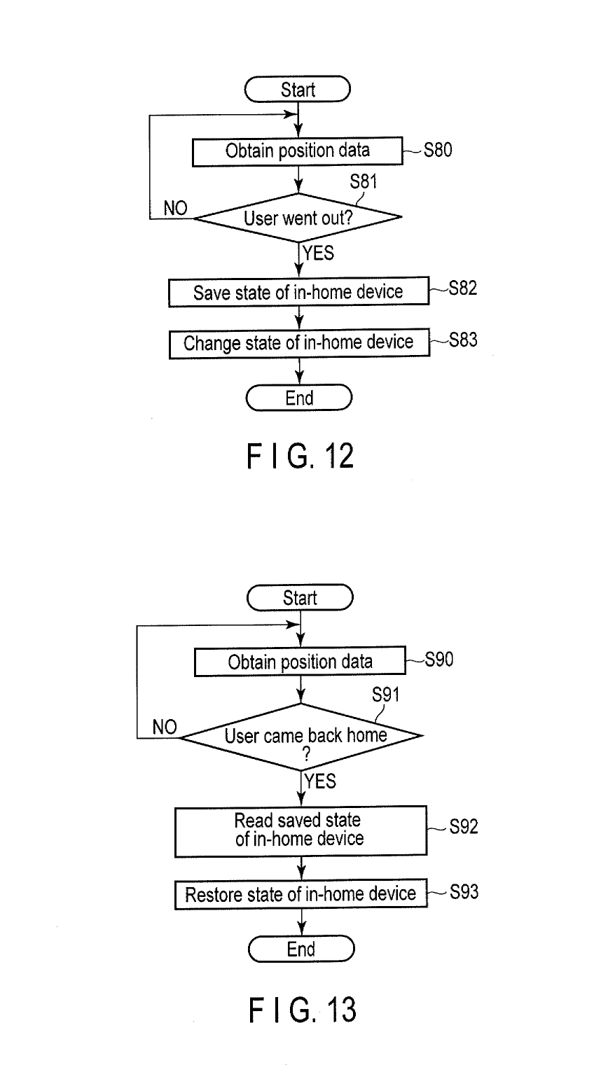

This specification explains steps of a state saving (changing) process when the user 11 goes out, referring to FIG. 12.

First, the remote control program 41 obtains the position data of the wearable device 10 by using the GPS and the like (step S80). The remote control program 41 determines whether or not the user 11 went out by comparing the obtained position data to the position data in the house (at home) (step S81). When the remote control program 41 determines that the user 11 did not go out (NO in step S81), the remote control program 41 waits for position data to be obtained again. When the remote control program 41 determines that the user 11 went out (YES in step S81), the remote control program 41 obtains the state of an external device in the house from external device 12 through communication between the wearable device 10 and external device 12 (step S82). Subsequently, the remote control program 41 changes the state of external device 12 by transmitting a corresponding control command to the external device in order to turn the unnecessary external device off (step S83).

After step S82, steps corresponding to step S20 and step S21 shown in FIG. 5 may be performed. After step S20 and step S21, step S83 is conducted. If step S20 and step S21 are performed in this manner in the state saving process shown in FIG. 12, the state of external device 12 is not changed in the following case: the remote control program 41 determines that the user went out in step S81, but the user who went out is not authorized to change the state of external device 12, or in other words, the biometric authentication of the user who went out fails.

When step S21 is performed in the state saving process shown in FIG. 12, the following case is possible: the user 11 goes out and does not wear or remove the wearable device 10. For example, the remote control program 41 may detect the acquisition of user's biological data by the biological data acquisition controller 35 as attachment of the wearable device 10 to the user 11 or removal of the wearable device 10 from the user 11. The remote control program 41 may detect the acquisition of user's biological data by the biological data acquisition controller 35 and the successful biometric authentication by the biometric authentication module 55 as attachment of the wearable device 10 to the user 11 or removal of the wearable device 10 from the user 11. Therefore, even in a case where the user 11 goes out and does not wear or remove the wearable device 10, step S83 can be performed after the user's biological data is obtained or after the user's biological data is obtained and the biometric authentication becomes successful by the biometric authentication module 55.

This specification explains steps of a state saving (changing) process when the user 11 comes back home, referring to FIG. 13.

First, the remote control program 41 obtains the position data of the wearable device 10 by using the GPS and the like (step S90). The remote control program 41 determines whether or not the user 11 came back home by comparing the obtained position data to the position data in the house (at home) (step S91). When the remote control program 41 determines that the user 11 has not got home (NO in step S91), the remote control program 41 waits for position data to be obtained again. When the remote control program 41 determines that the user 11 got home (YES in step S91), the remote control program 41 reads the external device state data stored (saved) in the storage 50 (step S92). Subsequently, the remote control program 41 restores the state of external device 12 by transmitting a corresponding control command to external device 12 in order to restore the state of external device 12 (step S93).

After step S91, steps corresponding to step S20 and step S21 shown in FIG. 5 can be performed. After step S20 and step S21 are conducted, step S92 is executed. If step S20 and step S21 are performed in this manner in the state saving process shown in FIG. 13, the state of external device 12 is not restored in the following case: the remote control program 41 determines that the user got home in step S91, but the user who got home is not authorized to change the state of external device 12, or in other words, the biometric authentication of the user who got home fails.

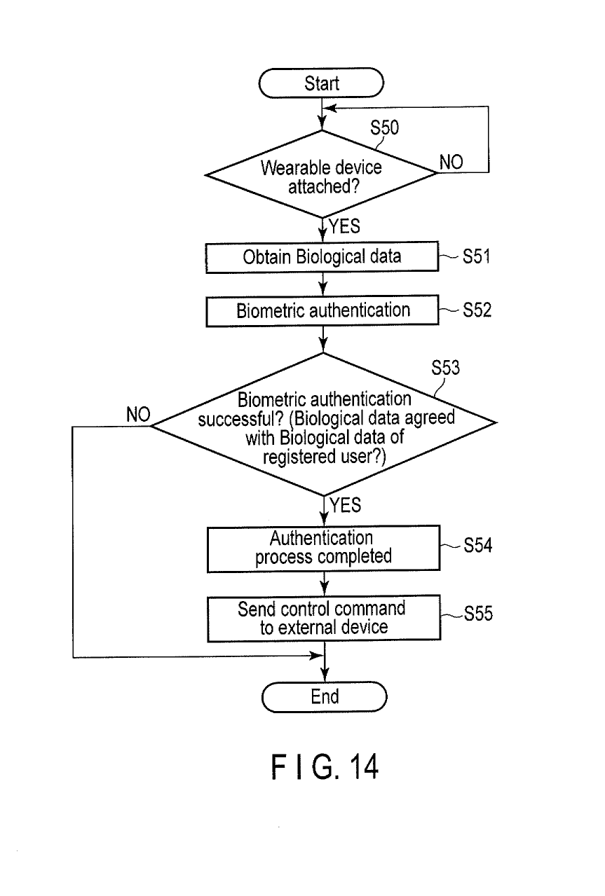

Now, this specification explains steps of a biometric authentication process which is executed by the wearable device 10, referring to FIG. 14.

First, a biometric authentication process is started upon detection of attachment or removal of the wearable device 10 by the attachment/removal detector 37 (as a trigger). The authentication module 52 determines whether or not the wearable device 10 is attached or removed (step S50). When attachment of the wearable device 10 is detected (YES in step S50), the biometric authentication module 55 obtains biological data from the biological data acquisition controller 35 (step S51). When attachment of the wearable device 10 is not detected (NO in step S50), the authentication module 52 waits for the attachment/removal detector 37 to detect attachment or removal of the wearable device 10 again.

Subsequently, the biometric authentication module 55 determines whether or not the biometric authentication is successful (step S53). Specifically, the biometric authentication module 55 compares the user's biological data stored in the storage 50 to the biological data obtained from the biological data acquisition controller 35. When the biometric authentication is not successful (NO in step S53), the biometric authentication process is terminated. When the biometric authentication is successful (YES in step S53), that is, when the user's biological data stored in the storage 50 corresponds to the biological data obtained from the biological data acquisition controller 35, the authentication module 52 determines that the user authentication is successful and the authentication process is completed (step S54). The authentication module 52 notifies the controller 51 that the user authentication is successful and the authentication process is completed. The controller 51 transmits a control command corresponding to the user who is successfully authenticated to external device 12 (step S55). For example, the control commands stored in the storage 50 may be associated with a plurality of users. Thus, the controller 51 is configured to transmit a control command corresponding to the user who is successfully authenticated to external device 12.

In a case where the buckle is closed while the wearable device 10 is not attached to the arm of the user 11, step S51 and the subsequent steps are performed. However, when the wearable device 10 is not attached to the arm of the user 11, the biological data acquisition controller 35 is not able to obtain biological data in step S51. In this case, biometric authentication is conducted in step S52, and the biometric authentication fails in step S53. Thus, no problem occurs in the biometric authentication process shown in FIG. 14. When biological data cannot be obtained in step S51, the biometric authentication process shown in FIG. 14 may be terminated.

As described above, in the present embodiment, the wearable device 10 is configured to detect either attachment to or removal from the human body, and to save control data indicating the content of the process which should be executed when attachment or removal is detected. When either attachment or removal of the wearable device 10 is detected, the wearable device 10 is configured to transmit a command (control command) for performing a process specified by the control data to at least one external device specified by the control data either when the attachment of the wearable device 10 is detected or when the removal of the wearable device 10 is detected. For example, the wearable device 10 is configured to transmit a command for setting at least one external device which is the control target specified by the control data to the state specified by the control data to at least one external device based on the control data. It is possible to solve the problem in which the user has to separately conduct operations of a plurality of information devices (external devices) at home. In addition, the user 11 can operate a plurality of information devices at home as a whole upon attachment of the wearable device 10, removal of the wearable device 10 or change in the body temperature of the user 11 (as triggers).

The wearable device 10 may have a part of the third function explained with reference to FIG. 10, etc. For example, a part of the third function refers to the function for saving the state of an external device present in a predetermined area upon detection of exit of the wearable device 10 from the predetermined area.

Now, this specification describes the aforementioned control commands in the present embodiment in more detail. A control command is a command for changing the state of an external device and is also a command for remotely controlling an external device. Specifically, a control command is a command for allowing the wearable device 10 to change the state (for example, an on-state or an off-state) of an external device selected in advance by the user 11 from external devices detected by using the Bluetooth module 36. Control data is assumed to include data indicating the content of control relative to an external device, or in other words, the content for changing the state of an external device. In this case, the remote control program 41 may convert data indicating the content of control relative to an external device into a control command and transmit the converted control command to the external device.

A control command may be a command obtained from an external device. For example, from the external device detected by the wearable device 10 through the Bluetooth module 36, the remote control program 41 obtains control data including the controllable content relative to the detected external device. The remote control program 41 saves the obtained control data. When attachment or removal of the wearable device 10 is detected, the remote control program 41 transmits a control command obtained based on the saved control data to a corresponding external device, for example, to the external device which transmitted the saved control data to the wearable device 10.

When no communication function is provided in the above wearable device 10, another device having a communication function may be employed. For example, the wearable device 10 may use a mobile phone having a communication function, communicate with the mobile phone and transmit a control command to an external device via the mobile phone.

The functions of the modules shown in FIG. 4 and FIG. 11 can be realized by software (computer programs). Therefore, an effect similar to that of the present embodiment can be easily realized by installing the software into a normal computer through a computer-readable storage medium in which the software is stored and executing the software.

The remote control program 41 explained in the present embodiment may be realized by hardware such as a dedicated LSI, a DSP or a microcomputer.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An electronic device comprising:
a first detector configured to detect either attachment of the electronic device to a human body or removal of the electronic device from a human body;
a memory configured to store control data; and
circuitry configured to execute a process for transmitting a command for performing a process specified by the control data to at least one external device specified by the control data either when the attachment of the electronic device is detected or when the removal of the electronic device is detected.

2. The electronic device of claim 1, wherein
the control data includes first control data indicative of content of a process to be executed when the attachment of the electronic device is detected and second control data indicative of content of a process to be executed when the removal of the electronic device is detected, and
the circuitry is configured to:
execute a process for transmitting a first command for setting at least one external device which is a control target specified by the first control data to a first state specified by the first control data when the attachment of the electronic device is detected; and
execute a process for transmitting a second command for setting at least one external device which is a control target specified by the second control data to a second state specified by the second control data when the when the removal of the electronic device is detected.

3. The electronic device of claim 1, further comprising a sensor configured to detect biological data of a user to which the electronic device is attached, wherein
the control data includes another type of control data indicative of content of a process to be executed when the biological data of the user is changed, and
the circuitry is configured to execute a process for transmitting a command for setting at least one external device specified by said another type of control data to a state specified by said another type of control data when change in the biological data of the user is detected during the attachment of the electronic device to the user.

4. The electronic device of claim 1, further comprising a second detector configured to obtain position data of the electronic device, wherein
the circuitry is configured to determine whether the electronic device is present in a first area in which the at least one external device is present based on the position data, and the circuitry is configured to enable a function for transmitting a command when the electronic device is present in the first area and to disable the function when the electronic device is present outside the first area.

5. The electronic device of claim 1, further comprising a sensor configured to obtain biological data of a user to which the electronic device is attached, wherein
the circuitry is configured to perform biometric authentication for determining whether the obtained biological data corresponds to registered biological data,
the circuitry is configured to enable a function for transmitting a command when the obtained biological data corresponds to the registered biological data, and to disable the function when the obtained biological data does not correspond to the registered biological data.

6. The electronic device of claim 1, further comprising a sensor configured to obtain biological data of a user to which the electronic device is attached, wherein
the first detector is configured to detect the attachment of the electronic device to the user or the removal of the electronic device from the user based on the obtained biological data.

7. The electronic device of claim 1, further comprising a second detector configured to obtain position data of the electronic device, wherein
the circuitry is configured to detect exit of the electronic device from a first area in which the at least one external device is present to outside of the first area or entry of the electronic device from outside of the first area into the first area based on the position data,
the control data includes another type of control data indicative of content of a process to be executed when the exit of the electronic device to the outside of the first area is detected, and
the circuitry is configured to:
execute a process for saving data indicative of a current state of at least one external device specified by said another type of control data and a process for transmitting a first command for setting the at least one external device specified by said another type of control data to a state specified by said another type of control data when the exit of the electronic device to the outside of the first area is detected; and
execute a process for transmitting a second command for restoring the at least one external device specified by said another type of control data to an original state based on the saved data when the entry of the electronic device into the first area is detected.

8. A method for remotely controlling an external device by an electronic device, the method comprising:
detecting either attachment of the electronic device to a human body or removal of the electronic device from a human body; and
transmitting a command for performing a process specified by control data to at least one external device specified by the control data either when the attachment of the electronic device is detected or when the removal of the electronic device is detected.

9. The method of claim 8, wherein
the control data includes first control data indicative of content of a process to be executed when the attachment of the electronic device is detected and second control data indicative of content of a process to be executed when the removal of the electronic device is detected, and
the transmitting comprises:
transmitting a first command for setting at least one external device which is a control target specified by the first control data to a first state specified by the first control data when the attachment of the electronic device is detected; and
transmitting a second command for setting at least one external device which is a control target specified by the second control data to a second state specified by the second control data when the when the removal of the electronic device is detected.

10. The method of claim 8, further comprising detecting biological data of a user to which the electronic device is attached, wherein
the control data includes another type of control data indicative of content of a process to be executed when the biological data of the user is changed, and
the transmitting comprises transmitting a command for setting at least one external device specified by said another type of control data to a state specified by said another type of control data when change in the biological data of the user is detected during the attachment of the electronic device to the user.

11. The method of claim 8, further comprising:
obtaining position data of the electronic device;
determining whether the electronic device is present in a first area in which the at least one external device is present based on the position data;
enabling a function for transmitting a command when the electronic device is present in the first area; and
disabling the function when the electronic device is present outside the first area.

12. The method of claim 8, further comprising:
obtaining biological data of a user to which the electronic device is attached;
performing biometric authentication for determining whether the obtained biological data corresponds to registered biological data;
enabling a function for transmitting a command when the obtained biological data corresponds to the registered biological data; and
disabling the function when the obtained biological data does not correspond to the registered biological data.

13. The method of claim 8, further comprising obtaining biological data of a user to which the electronic device is attached, wherein
the detecting includes detecting the attachment of the electronic device to the user or the removal of the electronic device from the user based on the obtained biological data.

14. The method of claim 8, further comprising:
obtaining position data of the electronic device;
detecting exit of the electronic device from a first area in which the at least one external device is present to outside of the first area or entry of the electronic device from outside of the first area into the first area based on the position data;
when the exit of the electronic device to the outside of the first area is detected:
saving data indicative of a current state of at least one external device specified by another type of control data; and
transmitting a first command for setting the at least one external device specified by said another type of control data to a state specified by said another type of control data, and
when the entry of the electronic device into the first area is detected, transmitting a second command for restoring the at least one external device specified by said another type of control data to an original state based on the saved data.
